(19) United States
(12) Patent Application Publication
(10) Pub. No.: US 2010/0036309 A1
(43) Pub. Date: Feb. 11, 2010

(54) LOW ABSORBENCY TAMPON PLEDGET AND METHOD OF TESTING

(75) Inventors: Robert T. Jorgensen, JR., Middletown, DE (US); Yinka Abdul, Newark, DE (US); Philip Burkhardt, Magnolia, DE (US)

Correspondence Address: MICHAUD-Kinney Group LLP, 306 INDUSTRIAL PARK ROAD, SUITE 206, MIDDLETOWN, CT, 06457, US

(73) Assignee: PLAYTEX PRODUCTS, LLC, Shelton, CT (US)

(21) Appl. No.: 12/400,070

(22) Filed: Mar. 9, 2009

Related U.S. Application Data
(60) Provisional application No. 61/035,622, filed on Mar. 11, 2008.

(51) Int. Cl. A61F 13/20

(52) U.S. Cl. 604/15; 604/385.17; 604/379

(57) ABSTRACT

An improved light duty tampon is defined by a quantity of an absorbent material arranged in substantially cylindrical form and having a lower dry bulk density, lower expansion values, and lower fluid absorption rates, as compared to similar absorbent capacity tampon pledgets. A method of testing a light duty tampon includes determining absorbent capacity, density, radial expansion width, and expansion rate of the tampon.

### CROSS REFERENCE TO RELATED APPLICATION

This application claims the benefits of U.S. Provisional Patent Application Ser. No. 61/035,622, filed Mar. 11, 2008, entitled “Lite Tampon Pledgets With Unique Density,” the content of which is incorporated herein by reference in its entirety.

### FIELD OF THE INVENTION

The present invention relates generally to an improved tampon pledget. More particularly, the present invention relates to an improved low absorbency tampon pledget having a reduced dry bulk density, reduced expansion value, and reduced fluid absorption rates, when compared to other similar tampons. The improved pledget also provides better leakage protection, placement control, and user comfort.

### BACKGROUND

Currently, tampon users have a choice of five FDA controlled product capacities (absorbency ranges): Lights (Lites)/Slim<6 grams (hereinafter referred to as “light duty”), Regular 6-9 grams, Super 9-12 grams, Super Plus 12-15 grams, and Ultra 15-18 grams. These tampons may have an applicator (such as cardboard or plastic) or may be inserted digitally. While the range of absorbent capacities of tampon pledgets covers capacity for menses flow of many women, a need for other sizes exists. A tampon's intended capacity and a user's changing/removal frequency vary widely. For example, some users may remove the tampon often (every 1-5 hours) while others change tampons anywhere from every 6-12 hours. Some users change as infrequently as once every 24 hours. Studies have shown that regardless of tampon usage time (1-24 hours), often the tampon has a fluid load of <6 grams.

The light duty tampons described in U.S. Pat. No. 6,682,513 included the following properties: an absorbent capacity of <6 grams (FDA requirement), a dry state width of <15 mm, and a wet expansion size of about 20 mm. Commercially available light duty tampons tend to be high density low capacity tampons. Measurable quantities that translate to leakage propensity and user comfort associated with light duty tampons, have not yet been determined.

### SUMMARY OF THE INVENTION

In one aspect, the present invention resides in an improved light duty tampon defined by a quantity of an absorbent material arranged in substantially cylindrical form and having a lower dry bulk density, lower expansion values, and lower fluid absorption rates, as compared to similar absorbent capacity tampon pledgets. These measureable quantities translate to better leakage protection, user placement, and user comfort.

The tampon of the present invention has the following tampon characteristics: (1) Low capacity as measured by Federal Register Part 801, 801.43, of <6 grams and preferably <5 grams; (2) a density profile where at least two measured areas are 0.20 g/cc or less; (3) a lower expansion rate where every area measured is 1.7 mm/min or less; and (4) a Delta expansion of 50% or less, at all three measured areas.

The present invention describes a light duty capacity tampon (absorbent capacity <6 grams) with unique properties differing from light duty tampons available commercially, in that the tampon is made with one or more absorbent materials that provide for a controlled amount of expansion width, rate, and delta. Since the amount of fluid absorbed by such a tampon is relatively low, the expansion value based on fluid absorbed is low. Therefore, the tampon has a lower density and higher surface area than the high density, low surface area commercially available light duty tampons to provide the desired protection. Thus, the increased size of the tampon of the present invention, allows the vaginal cavity to have more contact with the tampon. This results in bypass leakage protection, placement control and comfort.

In another aspect, the present invention resides in a method of testing a light duty tampon. In such a method, a light duty tampon is provided, and various attributes are determined. These attributes include, but are not limited to, absorbent capacity, density, radial expansion width, and expansion rate.

### BRIEF DESCRIPTION

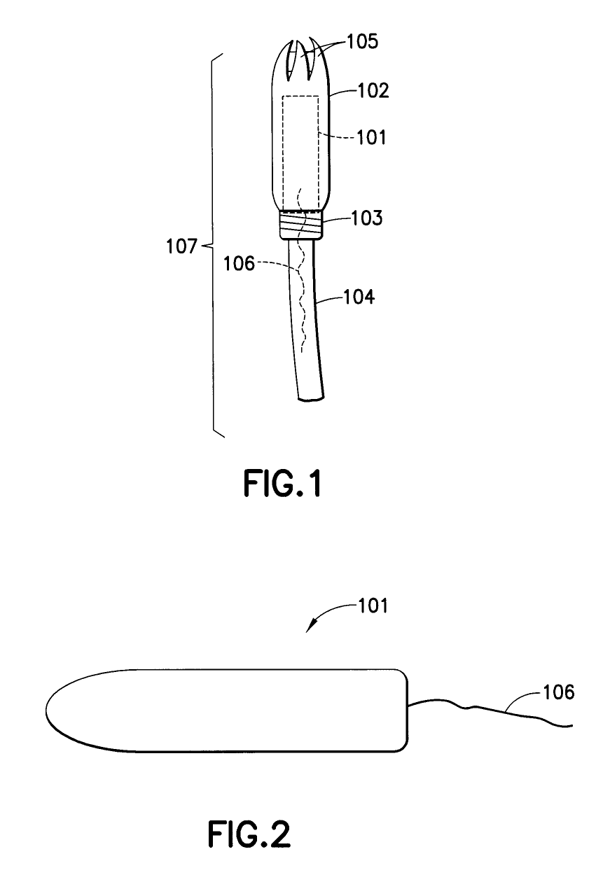

FIG. 1 is a front view of an applicator type tampon of the present invention;

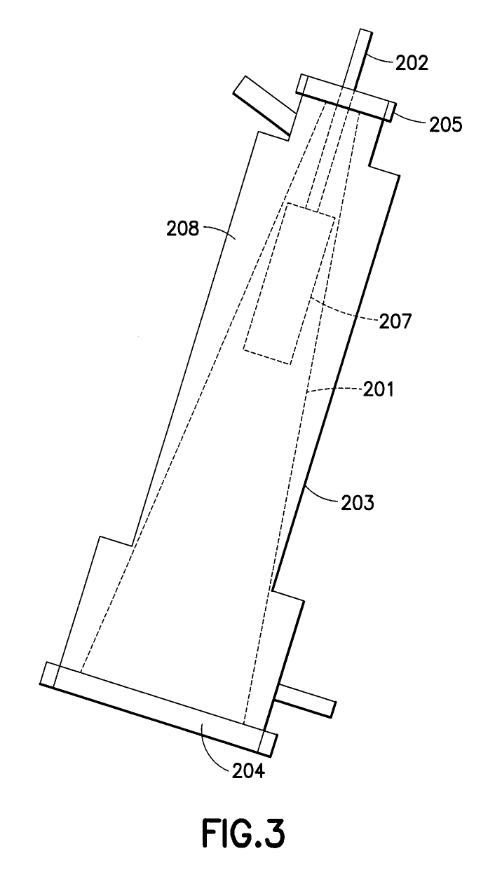

FIG. 2 is a schematic view of the tampon of FIG. 1;

FIG. 3 is a front view of the set up for the absorbent capacity test method;

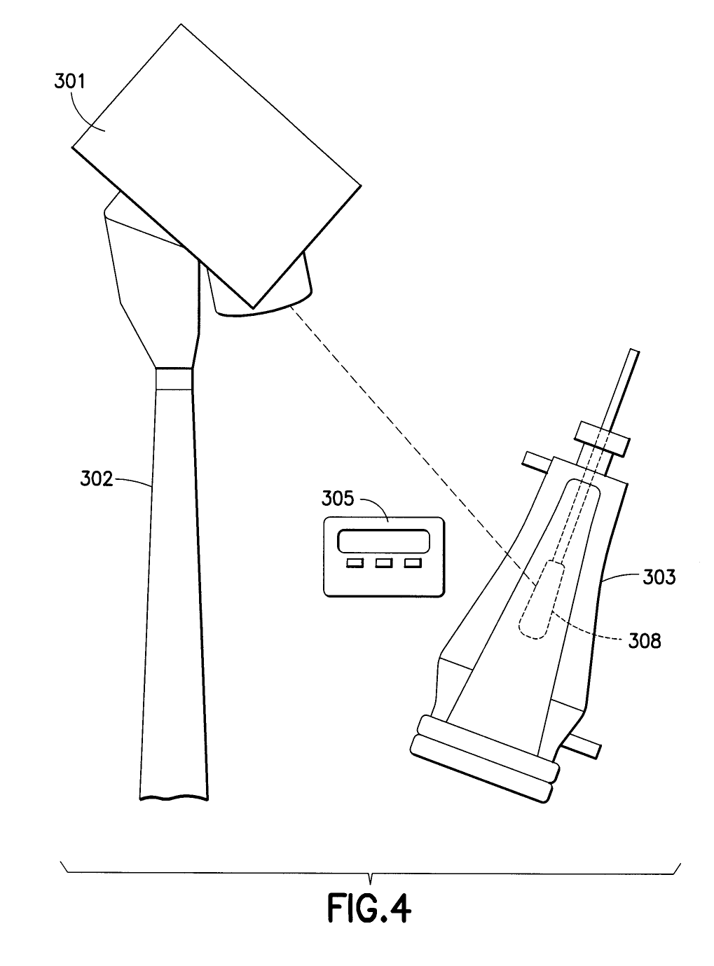

FIG. 4 is a front view of the set up for the radial expansion width and expansion rate test methods.

### DETAILED DESCRIPTION

The terms “tampon,” “pledget,” and “tampon pledget” are intended to be used interchangeably.

Consumer use testing demonstrates that a tampon's intended design capacity and women's tampon removal habits are very different, with many women removing tampons before the pledget reaches 6 grams of capacity. Light duty tampons provide the capacity required by the FDA (<6 grams), however, they may changed prematurely due to user fears of leakage, inadequate performance and discomfort. These qualities can be addressed by providing a tampon that focuses on measurable quantities such as: expansion rate, expansion delta, and ovality. The smaller size/appearance of commercially available light duty tampons, however, may lend itself to user anxiety about the tampon's ability to provide sufficient leakage protection. Furthermore, the high dry bulk density found in these tampons tends to be associated with a more rigid and therefore less comfortable tampon. By decreasing the light duty tampon's dry density and reducing overall mass, the resultant light duty tampon of the present invention has an overall size similar to that of a Regular absorbency tampon, a “Regular absorbency” tampon having a greater absorbency than a light duty tampon. The increased overall size of the light duty tampon of the present invention conveys security to users, while improving softness and general comfort. Furthermore, the lower density, lower mass tampon covers more area of the vaginal cavity as compared to other light duty tampons, which results in increased bypass leakage protection. Decreasing the dry bulk density also slows the rate of expansion and decreases the delta expansion value, providing increased comfort.

Light duty tampons of the present invention are designed to have: (1) lower absorbent capacity as compared to other grades of tampons (e.g., “Regulars” and the like); (2) higher surface area to capacity ratio that can reduce bypass leakage; (3) lower swell rate in response to fluid; and (4) good user placement control. These end use tampon qualities are based on measurable quantities of: absorbent capacity; radial expansion width; rate of expansion; expansion delta; dry density profile; and a tampon placement mechanism. Such measurable quantities translate tampon performance criteria into tangible measureable properties.

One embodiment of the present invention provides a low absorbent capacity tampon of <6 grams, and preferably <5 grams.

Another embodiment of the present invention provides a low absorbent capacity tampon with a density profile where at least two measured areas are 0.20 g/cc or less.

In a further embodiment of the present invention a low absorbent capacity tampon is provided with a lower expansion rate, where every area measured is 1.7 mm/min or less.

In yet another embodiment of the present invention a low absorbent capacity tampon is provided with a Delta expansion of 50% or less, at all three measured areas.

As used herein, the term “tampon” refers to any type of absorbent structure, which is fluid expanding, and that can be inserted into the vaginal canal, with or without an applicator, for the absorption of fluid therefrom. FIG. 1 illustrates the front view of an applicator type tampon. The tampon pledget 101 is housed within the barrel 102 of the applicator 107. A finger grip area 103 is located at the base of the barrel 102. A plunger 104 removably engages with the barrel 102 through the finger grip area 103. A string 104 may be connected to the tampon pledget 101 for removal from the vaginal cavity. The top of the barrel 101 has several petals 105 forming an opening for ejection of the pledget 101. Referring to FIG. 2, the tampon pledget 101 is a configuration of compressed absorbent material(s) arranged such that upon the absorption of up to about 6 grams of body fluid (e.g., menses), the material(s) of the pledget expand at a controlled rate and to a predetermined amount in the radial directions. The present invention is not limited in this regard, as the tampon pledget 101 may be defined by other configurations of absorbent material(s).

In any configuration of the tampon pledget 101 of the present invention, the absorbent material is selected such that the dry density thereof and the mass allows for the overall size of the tampon pledget 101 to be similar to tampons having greater absorbencies.

The tampon pledget measurable quantities include:

Absorbent Capacity: Currently, tampon users have a choice of five FDA controlled product absorbent capacities: Lites (light duty) <6 grams, Regular 6-9 grams, Super 9-12 grams, Super Plus 12-15 grams, and Ultra 15-18 grams. The guidelines regarding standard FDA Syngyna capacity are outlined in the Federal Register Part 801, §801.43 and testing was performed in accordance with U.S. Pat. No. 6,682,513 (herein incorporated by reference in its entirety). As Table 2 illustrates, the syngyna absorbent capacity for tampons of the present invention, are within the required <6 gram range.

Dry Bulk Density: The density of the tampon is measured at specific points along the longitudinal length of the tampon prior to exposure to fluid. The lower density profile contributes to a surface area that is immediately available for liquid absorption. Since the amount of fluid available for expansion is low, the density is adjusted to compensate. Thus the low capacity tampon of the present invention has a low dry bulk density, which results in a higher surface area.

Radial Expansion Width and Expansion Rate: Previously, faster expanding tampons were desired as they were thought to cover the vaginal cavity quickly, reducing the potential for bypass leakage. In this case, however, the amount of fluid is low and a larger beginning surface area is present, so the expansion rate does not need to be as rapid. The width of the tampon is measured at specific time intervals (t) of 0, 1, 2, 3 and 4 minutes, for example, at the top, bottom, and widest radial distances. The width is measured in mm. When t=0 minutes, the tampon pledget is dry and has not yet contacted fluid. At t=4 minutes, the tampon is wet and has been in contact with fluid for 4 minutes. It should be appreciated by those of skill in the art, that any time intervals may be used. For example, less frequent time intervals over a longer time period may be used when evaluating pledgets of higher absorbent capacity. The tampon pledget of the present invention has a larger diameter at the top and widest portions (at t=0 minutes) than commercially available tampon pledgets of the same absorbent capacity. This alleviates a potential tampon user's apprehension of bypass leakage by providing immediate coverage upon insertion. The expansion rate is measured in mm/minute and indicates the time it takes for the diameter of the widest part of the pledget to reach a certain width. The light duty tampon of the present invention expands slowly. A larger radial width when dry (at t=0 minutes), however, translates to less bypass leakage.

Expansion Delta: This term relates radial expansion width to expansion rate, meaning the change in the pledget dimensions over time. A high expansion delta accounts for a tampon pledget that blooms to a high degree during the tampon pledget's exposure to fluid. A tampon pledget of this nature is comparatively small at t=0 minutes and blooms quickly to cover the vaginal cavity to prevent bypass leakage, showing a high degree of change over time. The relatively large initial size of the light duty tampon of the present invention, however, provides a lower expansion delta. This property provides improved comfort and ease of placement for the user.

The formula for calculating the expansion delta percentage is as follows:

$$Expansion Delta = \frac{( Expansion Width @ t = 4 ) - ( Expansion Width @ t = 0 )}{( Expansion Width @ t = 0 )} * 100$$

### Examples

### Test Methods

Standard Syngyna Test (Absorbent Capacity): Testing was done in accordance with Standard FDA Syngyna capacity as outlined in the Federal Register Part 801, §801.43, as illustrated in FIG. 3. An un-lubricated condom 201, with tensile strength between 17-30 Mega Pascals was attached to the large end of a glass chamber 203 with a rubber band 204 and pushed through the small end using a smooth, finished rod. The condom was pulled through until all the slack was removed. The tip of the condom was cut off and the remaining end of the condom was stretched over the end of the tube and secured with a rubber band 205. A tampon 207 pre-weighed (to the nearest 0.01 gram) was placed within the condom membrane 201 so that the center of gravity of the tampon 207 was at the center of the chamber 203. An infusion needle (14 gauge) 202 was inserted through the septum created by the condom tip 201 until it contacted the end of the tampon 207. The outer chamber 208 was filled with water pumped from a temperature controlled water bath to maintain the average temperature of 27±1° C. The water was returned to the water bath.

The Syngyna fluid (10 grams sodium chloride, 0.5 grams Certified Reagent Acid Fuchsin, diluted to 1,000 milliliters with distilled water) was then pumped through the infusion needle 202 at a rate of 50 milliliters per hour. The test was terminated when the tampon 207 was saturated and the first drop of fluid exited the apparatus. The test was aborted if fluid was detected in the folds of the condom before the tampon 207 was saturated. The water was then drained and the tampon 207 was removed and immediately weighed to the nearest 0.01 grams. The absorbent capacity of the tampon was determined by subtracting its dry weight from the wet final weight. The condom 201 was replaced after 10 tests or at the end of the day during which the condom 201 was used in testing, whichever occurred first.

Density Testing: With Vernier Calipers, the diameter and length of the tampon was measured. Using several sample pledgets of the same lot number, the moisture content was determined by utilizing a moisture analyzer (Mettler Toledo HR73 Halogen Moisture Analyzer). The remaining tampons were weighed to the nearest 0.01 grams, correcting for moisture content. First the total pledget volume was measured by pouring approximately 1 cc of lab salt (sodium chloride crystals, reagent grade, obtained from VWR catalog number VWGY30-5, Lot#41044109) into the bottom of a calibrated graduated cylinder (Kimble Kimax 50 ml; ±0.4 ml). The whole pledget with the string removed was placed in the graduated cylinder on top of the 1 cc of lab salt. Then the remaining 9 cc of lab salt was poured over the pledget. The graduated cylinder was tapped several times until the displacement reading was stable. The displacement reading from the graduated cylinder was recorded. Then the pledget was removed and all excess salt was removed. Utilizing the “EdgeCraft” 662 Electric Slicer and Holder, each tampon was sliced into 0.25 inch (6.35 mm) segment series. The above displacement procedure was repeated for each of the segment series. The pledget density was then calculated using the following formula:

$$Pledget Density = \frac{Segmented Pledget Weight}{( displacement - 10 )}$$

Radial Expansion Width and Expansion Rate: The Standard FDA Syngyna capacity test as stated above according to the Federal Register Part 801, §801.43 was modified as illustrated in FIG. 4. An Olympus E510 Digital SLR or Nikon D50 camera 301 was attached with a Pro-Master 58 mm 1× Macro filter to a 58 mm Olympus lens. The camera automatically focused and flashed when the picture was taken. The camera was mounted on a tripod 302 at a 30-degree angle (60-degrees by protractor) parallel to the syngyna chamber 303. The focal point was the center mid point on a small calibrated rule 304 inside the syngyna chamber 303 and a timer 305 was placed alongside the sygyna chamber 303. Both are viewable through the camera lens while keeping the camera 301 as close as possible. Light was provided by two black lights 306, 307 mounted alongside and facing the syngyna chamber 303. Photographic contrast was controlled by pivoting the black lights 306, 307 until the desired contrast was achieved. The first picture taken was of the dry tampon 308 in the syngyna chamber 303 and this was marked as t=0 (dry). The syngyna fluid was changed to 10 grams sodium chloride, 0.5 grams Certified Reagent Acid Fuchsin, 3.50 g of Bonn Trace dye (a yellow/green fluorescent dye from Bonneau Dye Corporation, 10815 Briggs Road, Cleveland, Ohio 44111) diluted to 1,000 milliliters with distilled water. Photographs were taken at 1 minute intervals. The photographs were analyzed using Scion Image analysis software. Three measurements were recorded: (1) the top—approximately 5 mm from the upper most end of the tampon, (2) the bottom—approximately 7 mm from the bottom most edge of the tampon, and (3) several measurements were made to determine the widest radial diameter. Calibration measurements can be made on known cylinders.

$$Expansion Rate ( mm \text{/} minute ) = \frac{( ending width - starting width )}{Time}$$

Data from an in-vitro comparison using the above test methods, of a low dry density light duty tampon pledget of the present invention, and two different types of TAMPAX tampons, is illustrated in Tables 1 and 2. As Table 2 illustrates, the absorbent capacity for light duty tampon pledgets of the present invention (column A), as well as commercially available pledgets (columns B and C), are within the <6 gram range.

Table 3 provides radial expansion and expansion rate data for light duty tampons of the present invention. Test methods for Syngyna absorbency, the photographic and imaging methods and methods for determining the radial expansion are similar to those provided above. One difference is that defibrinated sheep's blood was used in these experiments. This was obtained from Innovative Research (Novi, Mich.).

Table 4 provides pledget dimensions and density information for dry pledgets of the present invention as compared to a competitive, commercially available pledget (B, identified in Table 2).

TABLE 1
Pledget Comparisons for New Light Duty Pledget Design

| | Top (mm) | | | Bottom (mm) | | | Widest (mm) | | |
|---|---|---|---|---|---|---|---|---|---|
| Time (min) | A | B | C | A | B | C | A | B | C |
| 0 | 12.5 | 11.5 | 12.2 | 11.6 | 12.7 | 11.1 | 13.3 | 12.2 | 11.4 |
| 1 | 14.9 | 16.6 | 14.4 | 12.4 | 15.5 | 12.7 | 17.1 | 19.3 | 13.7 |
| 2 | 16.4 | 18.2 | 14.4 | 13.8 | 19.7 | 13.2 | 18.6 | 22.5 | 14.9 |
| 3 | 17.7 | 18.7 | 15.7 | 15.0 | 21.7 | 15.0 | 19.5 | 24.2 | 17.1 |
| 4 | 17.7 | 20.5 | 17.6 | 15.9 | 23.0 | 16.7 | 19.9 | 25.8 | 18.6 |
| Delta | 42% | 78% | 44% | 37% | 81% | 51% | 50% | 111% | 63% |
| Expansion | | | | | | | | | |

TABLE 2
Radial Expansion Width and Expansion Delta Comparison

| | A | B | C |
|---|---|---|---|
| @ 4 min | (mm/min) | (mm/min) | (mm/min) |
| Top Rate | 1.32 | 2.24 | 1.34 |
| Bottom Rate | 1.07 | 2.59 | 1.39 |
| Widest Rate | 1.64 | 3.42 | 1.81 |
| | (g/cc) | (g/cc) | (g/cc) |
| String End | 0.19 | 0.27 | 0.19 |
| Center | 0.29 | 0.37 | 0.22 |
| Petal End | 0.20 | 0.29 | 0.27 |
| Ovality (oval | | | |
| expansion) | | | |
| Dry | 0.48 | 0.49 | 0.51 |
| Wet | 0.67 | 1.16 | 0.80 |
| Capacity | 4.3 g | 5.4 g | 5.1 g |

A = New Slimfit pledget design of the present invention
B = TAMPAX Pearl Lite ND
C = TAMPAX Cardboard Lites ND

TABLE 3
Radial Expansions and Expansion Rates for Pledgets of the
Present Invention (Light Duty) (Standard Syngyna Test Method,
except that Sheep's Blood was Used as the Test Fluid)

| | TOP | BOTTOM | WIDEST |
|---|---|---|---|
| | (mm) | (mm) | (mm) |
| Initial Width, mm, average | 10.36 | 10.31 | 11.65 |
| Width, average, mm, after 4 minutes | 12.49 | 12.65 | 14.30 |
| of expansion | | | |
| Average (delta) Expansion, % | 20.5% | 22.7% | 22.7% |
| Rate of Expansion, avg. over 4 min, | 0.53 | 0.59 | 0.66 |
| (mm/min) | | | |
| Std. Dev. Of Rate of Expansion, over | 0.30 | 0.29 | 0.33 |
| 4 min (mm/min) | | | |
| Number of Tampon Samples tested/ | 5 | 5 | 5 |
| measured | | | |

TABLE 4
Dry Pledget Dimensions and Densities for Light Duty Tampons

| Pledget Measurements | Present Invention (A) | Comparative (B) |
|---|---|---|
| Pledget weight, excluding | 1.119 | 1.435 |
| string, g | | |
| Length (inches) | 1.630 | 1.444 |
| Width (inches) | 0.420 | 0.429 |
| Pledget Density, g/cc | 0.302 | 0.420 |

Although this invention has been shown and described with respect to the detailed embodiments thereof, it will be understood by those of skill in the art that various changes may be made and equivalents may be substituted for elements thereof without departing from the scope of the invention. In addition, modifications may be made to adapt a particular situation or material to the teachings of the invention without departing from the essential scope thereof. Therefore, it is intended that the invention not be limited to the particular embodiments disclosed in the above detailed description, but that the invention will include all embodiments falling within the scope of the following claims.

What is claimed is:

1. A tampon, comprising:
a compressed absorbent material arranged in substantially cylindrical form having an absorbent capacity of up to about 6 grams of liquid;
wherein a dry density of said absorbent material arranged in substantially cylindrical form is about 0.20 grams/cc or less; and
wherein said dry density of said absorbent material is substantially uniform through said quantity of said absorbent material.

2. The tampon of claim 1, wherein a weight of said absorbent material arranged in substantially cylindrical form is about 1.1 grams to about 1.3 grams.

3. The tampon of claim 1, wherein a length of said absorbent material arranged in substantially cylindrical form is greater than about 1.5 inches.

4. The tampon of claim 1, wherein a rate of expansion of said absorbent material over a measured time period is about 1.7 mm/min or less.

5. The tampon of claim 1, wherein an average delta expansion of said absorbent material is about 50% or less.

6. The tampon of claim 1, further comprising an applicator, wherein said applicator includes a plunger and a barrel.

7. The tampon of claim 1, wherein said rate of expansion of said absorbent material at said widest portion is greater than said rate of expansion at said first end and said second end.

8. A method of testing a light duty tampon, said method comprising the steps of:
providing a tampon having an absorbency of up to about 6 grams of liquid;
determining a density of said tampon;
determining a radial expansion width of said tampon; and
determining an expansion rate of said radial expansion of said tampon.

9. The method of claim 8, wherein said step of determining said density of said tampon comprises,
dividing said tampon into a plurality of equally sized sections,
measuring a volume of each equally sized section, and
calculating said density of said tampon by dividing a mass of reach of said equally sized sections by a total volume displacement of each equally sized section.

10. The method of claim 9, further comprising measuring a moisture content of each of said equally sized sections and correcting for said moisture content.

11. The method of claim 8, wherein said step of determining said expansion rate of said tampon comprises,
performing absorbent capacity testing of said tampon,
photographing said step of performing absorbent capacity testing,
analyzing photographs taken using image analysis software, and
calculating an expansion rate by determining a difference in width per unit time.

12. The method of claim 11, wherein said step of analyzing said photographs comprises obtaining width measurements at two or more places along a length of said tampon.

13. The method of claim 11, wherein said step of performing absorbent capacity testing of said tampon is in accordance with Syngyna testing procedures using a dye solution.

14. The method of claim 11, wherein said step of performing absorbent capacity testing of said tampon is in accordance with Syngyna testing procedures using defibrinated sheep's blood.
